(19) United States
(12) Patent Application Publication
(10) Pub. No.: US 2018/0271475 A1
(43) Pub. Date: Sep. 27, 2018

(54) SYSTEM FOR CONNECTION OF AN ELECTRONIC STETHOSCOPE TO A COMMUNICATION DEVICE

(71) Applicant: David J. Cooper, Exton, PA (US)

(72) Inventor: David J. Cooper, Exton, PA (US)

(21) Appl. No.: 15/722,331

(22) Filed: Oct. 2, 2017

Related U.S. Application Data
(60) Provisional application No. 62/403,252, filed on Oct. 3, 2016.

(51) Int. Cl. A61B 7/04; H03H 7/06; H03H 7/01; A61B 5/00

(52) U.S. Cl. CPC A61B 7/04; A61B 5/0022; H03H 7/0153; H03H 7/06

(57) ABSTRACT

A system for providing stethoscopic information to a remote user, the system comprising a communication device capable of receiving and outputting audible sounds and an electronic stethoscope for detecting and outputting audible patient sounds. The system further comprises a capacitive voltage divider circuit coupled to and disposed between the electronic stethoscope and the communication device, wherein the capacitive voltage divider circuit is configured to obtain an input voltage signal from the electronic stethoscope and reduce the input voltage signal to an output voltage signal suitable for access by the communication device.

This patent application claims the benefit of the priority of U.S. provisional application Ser. No. 62/403,252 filed 3 Oct. 2016 in the name of Dr. David J. Cooper and entitled “System for Connection of an Electronic Stethoscope to a Communication Device.” The priority of the '252 application is claimed under 35 USC 120.

### STATEMENT REGARDING FEDERAL FUNDING FOR THIS INVENTION AND TECHNOLOGY

Not applicable.

### FIELD OF THE INVENTION

This invention relates to electronic stethoscopes. More particularly, the invention relates to a system for connecting an electronic stethoscope to a communication device such as a smartphone or tablet computer.

### BACKGROUND OF THE INVENTION

Telemedicine is a term generally used to describe the use of telecommunication and information technology by physicians and other medical personnel to provide remote health care to patients. With the worldwide proliferation of smartphones, tablet computers, and other advanced personal computing and communication devices, telemedicine is increasingly seen as a viable alternative to conventional clinical patient visits. Patients may be virtually examined by a healthcare provider through videotelephony, voice-only telephony, image sharing, etc., which may save the patient both the time and cost associated with a clinical visit, and may afford the healthcare provider a more efficient means to determining a diagnosis.

While visual and/or verbal communication between medical personnel and patients certainly allows for effective care in many situations, there are instances where the healthcare provider may need more information prior to providing an accurate diagnosis and treatment plan. For instance, in a clinical setting, the healthcare provider will often utilize a stethoscope to listen to the patient's heart, lung, and/or bowel sounds. However, such non-visual and non-verbal information is generally not obtainable via current telemedicine methods, thereby limiting the efficacy of telemedicine in many instances.

Accordingly, this document describes devices and methods that are intended to addresses the issued discussed above and/or other issues.

### SUMMARY OF THE INVENTION

In at least one aspect, the present disclosure provides a system for providing stethoscopic information to a remote user, the system comprising a communication device capable of receiving and outputting audible sounds and an electronic stethoscope for detecting and outputting audible patient sounds. The system further comprises a capacitive voltage divider circuit coupled to and disposed between the electronic stethoscope and the communication device, wherein the capacitive voltage divider circuit is configured to obtain an input voltage signal from the electronic stethoscope and reduce the input voltage signal to an output voltage signal suitable for access by the communication device.

According to an aspect of the present disclosure, the capacitive voltage divider circuit comprises two capacitors coupled in series. A first capacitor may have a capacitance value between 1 μF-10 μF, and a second capacitor may have a capacitance value between 10 μF-22 μF.

In another aspect of the present disclosure, wherein the communication device is a smartphone device. Furthermore, the capacitive voltage divider circuit may also comprise a resistor, wherein the resistor is configured to turn off an internal microphone of the smartphone device.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is top view of a system for connecting an electronic stethoscope to a communication device in accordance with an aspect of the disclosure.

FIG. 2 is a schematic view of a capacitive voltage divider circuit in accordance with an aspect of the disclosure.

FIG. 3 is a view of a circuit board of the capacitive voltage divider in accordance with an aspect of the disclosure.

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

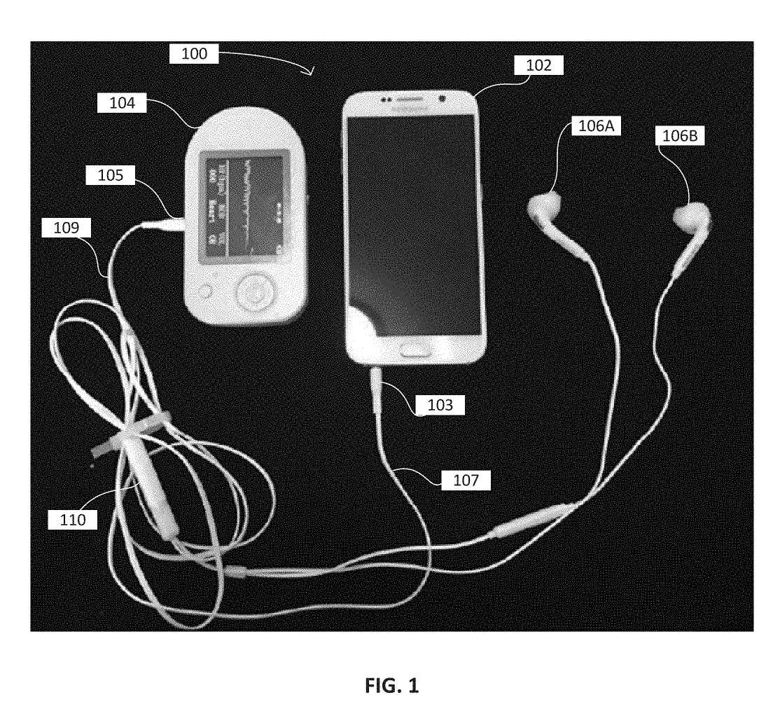

Referring to FIG. 1, an system 100 for connecting an electronic stethoscope to a communication device in accordance with an aspect of the present disclosure is illustrated. System 100 comprises a wireless communication device 102, such as a smartphone. It is to be understood that communication device 102 may alternatively be any device capable of remote communication, such as a tablet computer, personal computer, etc. Coupled to communication device 102 is an electronic stethoscope 104. Electronic stethoscope 104 is capable of detecting both low-frequency and high-frequency sounds, such as a patient's heartbeat, lung/breathing sounds, bowel sounds, etc.

Typically, electronic stethoscopes are used by a healthcare provider to directly listen to various internal sounds of the patient during a face-to-face patient visit. However, in accordance with an aspect of the present disclosure, electronic stethoscope 104 is capable of being electronically coupled to communication device 102 so as to transmit patient sounds captured by the patient or caretaker near the patient via the electronic stethoscope 104 to a remote healthcare provider via wireless communication. Specifically, system 100 comprises a cable 109 leading from an interface plug 105 connected to an input/output (I/O) port of the electronic stethoscope 104, while a cable 107 leads from an interface plug 103 connected to an I/O port of the communication device 102. Respective cables 107, 109 each connect to a capacitive voltage divider circuit housing 110, the operation of which will be further described below. A pair of headphone earbuds 106A, 106B may be utilized by the patient to listen to the output of the electronic stethoscope 104 and improve communication with the healthcare provider. However, use of earbuds 106A, 106B by the patient is optional. Furthermore, while electronic stethoscope 104 is shown as having a digital display screen and a user interface, it is to be understood that electronic stethoscope 104 may be configured more simply such that no display and/or user interface is necessary.

As noted above, the output signal of electronic stethoscope 104 is sent to communication device 102 through capacitive voltage divider circuit housing 110. Most communication devices (such as smartphones) can only be accessed by external voltage signals having a peak voltage of about 20-30 mV. However, most external devices (such as conventional electronic stethoscopes) are not specifically designed for integration with such communication devices and, thus, produce output signals having a much higher voltage, such as 50-600 mV. Accordingly, there is a need to reduce the output voltage signal provided to the communication device such that the signal is at or below the device's acceptable peak input voltage. In the case of some external devices (such as electronic musical instruments), such a reduction in output voltage signal is achieved through the use of a resistive voltage divider, which provides a reduction in voltage across the full frequency range of the signal. However, such a reduction in signal strength across the full frequency range is undesirable in electronic stethoscopes, as the low frequency range (e.g., 60-1200 Hz) must be preserved for the acquired internal sounds to be effectively transmitted in real time to a medical professional via the communication device.

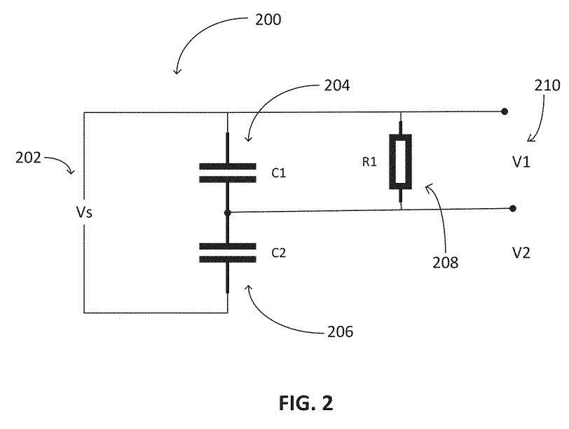

In order to preserve (and even enhance) the low frequency range of the output voltage system, a capacitive (rather than resistive) voltage divider circuit may be utilized in accordance with an aspect of the present disclosure, with the capacitive voltage divider circuit being housed in the capacitive voltage divider circuit housing 110 shown in FIG. 1. Referring to FIG. 2, a schematic diagram of an example capacitive voltage divider circuit 200 is shown. Circuit 200 comprises an input voltage 202, a first capacitor 204, and a second capacitor 206, wherein first capacitor 204 and second capacitor 206 are disposed in series. An optional resistor 208 may be disposed in parallel with capacitors 204, 206. In some communication devices (such as some models of smartphones), a resistor 208 may be utilized to turn off an internal microphone of the device. Resistor 208 may be, for example, a 1.8 K resistor. However, it is to be understood that resistor 208 does not affect the reduction in voltage and would not necessarily be present in every configuration and embodiment.

As the input voltage 202 passes through capacitors 204, 206, the voltage is reduced to form an output voltage 210, which is output at a level suitable for input into a communication device. The amount of voltage reduction is dependent upon the capacitance values of the

$$V 1 = Vs * \frac{C 1}{C 1 + C 2} .$$

capacitors 204, 206 and is determined by the equation Example capacitance values of first capacitor 204 may be 1 μF-10 μF, while example capacitance values of the second capacitor 206 may be 10 μF-22 μF. However, it is to be understood that other capacitance values for each of capacitors 204, 206 are possible and are within the scope of the present disclosure.

While circuit 200 allows for a reduced output voltage 210, the capacitors 204, 206 also act to filter out high-frequency signals. Accordingly, only signals in a low-frequency range (e.g., 60-1200 Hz) are output. As noted above, such low-frequency output is desirable in devices such as electronic stethoscopes, as heart, lung, and bowel sounds are generally low-frequency sounds. Thus, circuit 200 not only reduces the output voltage, but also enhances the output signal by way of high-frequency filtration.

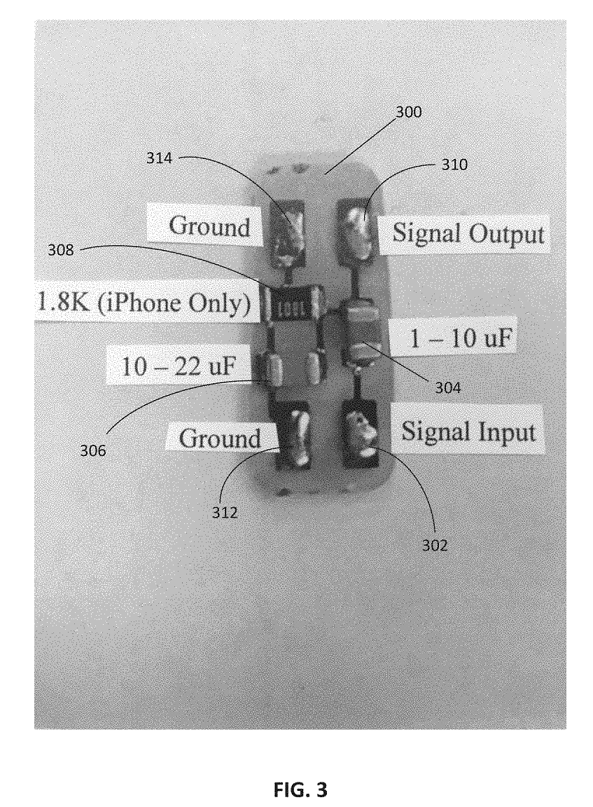

Referring to FIG. 3, a view of a capacitive voltage divider circuit board 300 in accordance with an aspect of the disclosure is shown. Circuit board 300, like circuit 200, comprises a signal input 302, a first capacitor 304, and a second capacitor 306 in series with the first capacitor 304. Again, an optional resistor 308 may be provided for shutting off the internal microphone of a connected communication device. A signal output 310 is also provided to output a reduced voltage signal to a communication device. Respective ground connections 312, 314 are also provided. As with circuit 200 described above, circuit board 300 may be housed within the capacitive voltage divider circuit housing 110 in-line between the electronic stethoscope and communication device. However, circuit board 300 may be housed in alternative locations, such as integrated within the housing of the electronic stethoscope or the housing of the communication device.

By utilizing the capacitive voltage divider circuit configuration(s) described above, aspects of the present disclosure allow for interface of an electronic stethoscope to a communication device (e.g., a smartphone) such that an enhanced signal may be transmitted from a patient to a healthcare provider in a remote location.

As discussed above and from the foregoing description of the exemplary embodiments of the invention, it will be readily apparent to those skilled in the art to which the invention pertains that the principles and particularly the structures disclosed herein and the methods of use thereof can be used for applications other than those specifically mentioned. All such applications of the invention are intended to be covered by the appended claims unless expressly excluded therefrom.

The invention may be embodied in other specific forms without departing from the spirit or essential characteristics of the invention. The disclosed embodiments are therefore to be considered in all respects as illustrative and not restrictive with the scope of the invention being indicated by the appended claims rather than by the foregoing description. All changes which come within the meaning and range of equivalency of the claims are therefore intended to be embraced therein.

As used in the claims herein, the term “comprising” means “including” while the term “consisting of” means “including so much and no more” and the term “consisting essentially of” means including the recited elements and those minor accessories required and known to be used in the art to facilitate the invention as claimed. The scope of the invention is, therefore, indicated by the appended claims rather than by the foregoing description and all changes which come within the range of equivalency of the claims are to be considered to be embraced within the scope of the claims.

What is claimed is:

1. A system for providing stethoscopic information to a remote user, the system comprising:
a) a communication device capable of receiving and outputting audible sounds;
b) an electronic stethoscope for detecting and outputting audible patient sounds; and
c) a capacitive voltage divider circuit coupled to and disposed between the electronic stethoscope and the communication device, wherein the capacitive voltage divider circuit is configured to obtain an input voltage signal from the electronic stethoscope and reduce the input voltage signal to an output voltage signal suitable for access by the communication device.

2. The system of claim 1, wherein the capacitive voltage divider circuit comprises two capacitors coupled in series.

3. The system of claim 2, wherein a first capacitor has a capacitance value between 1 μF-10 μF, and a second capacitor has a capacitance value between 10 μF-22 μF.

4. The system of claim 1, wherein the communication device is a smartphone device.

5. The system of claim 4, wherein the capacitive voltage divider circuit further comprises a resistor, wherein the resistor is configured to turn off an internal microphone of the smartphone device.

6. A system for providing stethoscopic information to a remote user, consisting of:
a) a communication device capable of receiving and outputting audible sounds;
b) an electronic stethoscope for detecting and outputting audible patient sounds; and
c) a capacitive voltage divider circuit coupled to and disposed between the electronic stethoscope and the communication device configured to obtain an input voltage signal from the electronic stethoscope and reduce the input voltage signal to an output voltage signal suitable for access by the communication device.
